(19) United States
(12) Patent Application Publication
(10) Pub. No.: US 2019/0185438 A1
(43) Pub. Date: Jun. 20, 2019

(54) SALTS AND POLYMORPHS OF ESREBOXETINE

(71) Applicant: Tonix Pharmaceuticals Holding Corp., New York, NY (US)

(72) Inventors: Seth Lederman, New York, NY (US); Bruce Daughtery, Mendham, NJ (US)

(21) Appl. No.: 15/833,973

(22) Filed: Dec. 6, 2017

Related U.S. Application Data
(60) Provisional application No. 62/430,863, filed on Dec. 6, 2016.

(51) Int. Cl. C07D 265/30; C07C 57/15

(52) U.S. Cl. CPC C07D 265/30; C07B 2200/13; C07C 57/15

(57) ABSTRACT

Disclosed herein are salts and polylmoprhs of (2S)-2-[(S)-(ethoxyphenoxy)phenylmethyl]morpholine (esreboxetine) as shown in Formula I:

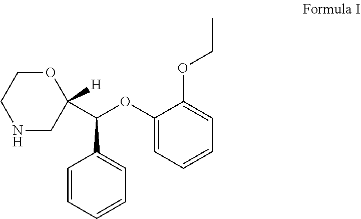 [SMILES: CCOc1ccccc1O[C@@H](c1ccccc1)[C@@H]1CNCCO1]

wherein an acid is selected from adipic, L-ascorbic, L-aspartic, fumaric, glycolic, hydrochloric, maleic, mucic, phosphoric, sulfuric, and thiocyanic acid.

### CROSS-REFERENCE TO RELATED APPLICATIONS

This application claims priority under 35 U.S.C § 119, based on U.S. Provisional Application Ser. No. 62/430,863, filed on 6 Dec. 2016, and entitled, “Salts and Polymorphs of Esreboxetine.”

### FIELD OF THE INVENTION

The present disclosure relates to various crystalline salts of esreboxetine, including new polymorphic forms of esreboxetine fumarate, methods of making the salts and polymorphic forms, and pharmaceutical compositions comprising them.

### BACKGROUND OF THE DISCLOSURE

Fibromyalgia is a chronic condition characterized by widespread pain, tenderness, fatigue, sleep disturbance, and psychological distress. (Arnold 2012). According to the preliminary diagnostic criteria for fibromyalgia compiled by the American College of Rheumatology (ACR), a diagnosis of fibromyalgia can be made when levels of the Widespread Pain Index (WPI) and Symptom Severity Scale (SSS) are sufficiently high (WPI≥7 and SSS≥5, or WPI is 3-6 and SSS≥9) (Walitt 2015). The WPI is a 0-9 count of painful non-articular body regions and the SSS is a 0-12 measure of symptom severity that includes fatigue, sleep and cognitive problems (Walitt 2015). Fibromyalgia is associated with increased rates of depression and other mental illnesses in addition to other comorbid conditions such as myocardial infarction, hypertension, and diabetes (Walitt 2015). The condition affects approximately 2% of the adult population in the US and worldwide prevalence estimates in adults range from 0.5% to 5.0% (Arnold 2012). The prevalence of fibromyalgia is considerably higher in women (3.4%) than in men (0.5%) (Arnold 2012). The range and severity of the symptoms associated with the condition result in a diminished quality of life (Arnold 2012).

Although the precise pathophysiology of the condition remains unknown, evidence suggests that dysregulation of serotonin and norepinephrine neurotransmission in descending analgesic systems in the brain and spinal cord mediate the pain associated with fibromyalgia (Arnold 2010). Tricyclic antidepressants that increase serotonin- and norepinephrine-mediated transmission are currently used in the treatment of fibromyalgia and have shown moderate efficacy against pain, fatigue, and sleep disturbances (Arnold 2000). Studies of the serotonin-norepinephrine reuptake inhibitors duloxetine and milnacipran show that selectively increasing serotonin and norepinephrine neurotransmission can reduce pain and other symptoms of fibromyalgia, as well as improve function and quality of life (Arnold 2004; Arnold 2005; Gendreau 2005). Selective serotonin reuptake inhibitors, however, have been reported to be less reliably effective in reducing pain associated with fibromyalgia than are dual serotonin-norepinephrine reuptake inhibitors (Anderberg 2000; Arnold 2002; Goldenberg 1996). In fact, evidence suggests that serotonin has pronociceptive and antintociceptive actions in descending pain-modulatory pathways in the brain and spinal cord (Millan 2002). Norepinephrine, in contrast, is thought to have predominately pain-inhibitory activity in these descending pain pathways (Millan 2002).

Esreboxetine, or (2S)-2-[(S)-(2-ethoxyphenoxy)-phenylmethyl]morpholine, is a highly selective norepinephrine reuptake inhibitor (SNRI). It is the active (S,S)-(+)-enantiomer of racemic reboxetine, a compound that was developed by Pharmacia & Upjohn (now Pfizer, Inc.). Table 1 shows the binding affinities of reboxetine as well as the S and R enantiomers to the norepinephrine transporter (NET), the serotonin transporter (SERT) and the ratio of NET/SERT of those binding affinities. In 1997, it was launched in Europe as a treatment for depression; however, it has been reported to have antinociceptive effects in preclinical pain models (Arnold 2010). Though few studies in the treatment of fibromyalgia have been published, the antinociceptive effect of norepinephrine reuptake inhibitors has been reported in preclinical pain models (Fishbain 2000; Bohn 2000).

TABLE 1
Binding Affinities of reboxetine and its enantiomers to NET and SERT

| | NET | SERT | Selectivity of Kᵢ |
|---|---|---|---|
| Compound | Kᵢ (nM) | Kᵢ (nM) | NET/SERT |
| (+/−)-Reboxetine | 1.1 | 129 | 124 |
| (+)-(S,S)-Reboxetine | 0.2 | 2900 | 14500 |
| (−)-(R,R)-Reboxetine | 7.0 | 104 | 15 |

Other targets with Kᵢ > 10000 include dopamine reuptake (DR1), muscarinic (M1, M2, M3, M4, M5), adrenergic (a1, a2, b1, b2), dopaminergic (D1, D2, D3, D4), serotinergic (5-HT1A, 5-HT2A, 5-HT3, 5-HT4, 5-HT6, 5-HT7), adenosine (A1, A2), benzodiazepine, L-type calcium channels), histaminergic (H1, H2), melatonin, NMDA, neurokinin NK1, nicotininc (a3, a4, a7), sigma, MAO-A, MAO-B, NOS, tyrosine hydroxylase and xanthine oxidase. Baldwin DS, Buis C, Carabal E (2000) Rev Contemp Pharmacother 11: 321. Hajos M, Fleishaker J C, Filipiak-Reisner J K, Brown M T, Wng EHF (2004) CNS Drug Rev 10: 23.

The efficacy of esreboxetine in the treatment of fibromyalgia has been shown in two randomized, double-blind, placebo controlled studies, one Phase 2 and one Phase 3, both sponsored by Pfizer, Inc.

In the Phase 2 study, Arnold et al., (2010), sought to assess the efficacy and safety profile of esreboxetine in the management of fibromyalgia. It was a multicenter, randomized, placebo-controlled study in patients 18 years of age and older who met the ACR criteria for fibromyalgia. Patients were randomized to receive esreboxetine or placebo for eight weeks, followed by one week follow-up period. Dosing was started at 2 mg/day and was increased by 2 mg/day every 2 weeks until a dose of 8 mg/day or the maximum tolerated dose was attained. The primary efficacy outcome was change from baseline to week 8 in weekly pain scores as derived from ratings on the 11-point scale. Additional primary efficacy outcomes included changes in Fibromyalgia Impact Questionnaire (FIQ) total score and Patient Global Impression of Change (PGIC). Following the 8-week trial for patients with fibromyalgia, it was concluded that esreboxetine was associated with statistically significant reductions in pain scores compared to placebo. Esreboxetine was also associated with improvements in outcomes relevant to fibromyalgia, including the PGIC, function, and fatigue (Arnold 2010). In addition, the drug was generally well-tolerated since there was little difference in the number of patients who discontinued treatment in the esreboxetine and placebo groups, and relatively few patients discontinued due to adverse events (8.2% and 2.3%, respectively) (Arnold 2010).

In the Phase 3 study (Arnold et al., 2012) the objective was to evaluate the efficacy, tolerability, and safety of multiple-fixed doses of esreboxetine for the treatment of fibromyalgia. Patients meeting ACR criteria for fibromyalgia were randomized to receive esreboxetine at doses of 4 mg/day (n=277), 8 mg/day (n=284), or 10 mg/day (n=283) or matching placebo (n=278) for 14 weeks. The primary efficacy outcomes included weekly mean pain score and the FIQ total score at week 14. Secondary efficacy measures included scores for the PGIC, the Global Fatigue Index (GFI), and the 36-item Short-Form health survey (SF-36; physical function scale only) at week 14. Following the 14-week trial patients that had received esreboxetine at all doses demonstrated significant improvement in the pain score (P≤0.025), the FIQ score (P≤0.023), and the PGIC score (P≤0.007) compared to placebo (Arnold 2012). In addition, patients receiving 4 mg/day and 8 mg/day of esreboxetine showed significant improvement in GFI score compared to placebo (P=0.001). Again, the study concluded esreboxetine was generally well tolerated and was associated with significant improvements in pain, FIQ, PGIC, and fatigue scores compared with placebo (Arnold 2012). The lack of a dose-response relationship in both the efficacy and safety analyses suggested that esreboxetine at a dosage of 4 mg/day would offer clinical benefit with the least risk of drug exposure. Table 2 provides a summary of the results for various endpoints at a dose of 4 mg/day (Arnold 2012).

TABLE 2
Efficacy of Esreboxetine in Fibromyalgia in Phase 3 Study
(Pfizer). Summary of Various Endpoints, 4 mg/day

| Parameter | Score | p value |
|---|---|---|
| Pain | −0.74* (10-point scale) | <0.001 |
| FIQ | −7.12* (100-point | <0.001 |
| | scale) | |
| GFI | −0.64* (10-point scale) | <0.001 |
| PGIC | 41.7%** | 0.002 |

FIQ = Fibromyalgia Impact Questionnaire;
GFI = Global Fatigue Index;
PGIC = Patient's Global Impression of Change;
*Treatment difference;
**much improved or very much improved.
Arnold L M, Hirsch I, Sanders P, Ellis A, Hughes B (2012) Arthr Rheum 64: 2387

To date, the US FDA has approved three pharmaceutical drugs for the treatment of fibromyalgia. These drugs include pregabalin (approved, June 2007), duloxetine (approved, June 2008), and milnacipran (approved, January 2009). However, in the seven years since milnacipran was approved, no additional drugs have gained FDA approval for the treatment of fibromyalgia. What's more, even though the clinical efficacy of the current therapies may be statistically superior to placebo, their small effect size may render them of little clinical importance (Blumenthal 2016). Non-pharmacological treatment methods, such as cardiovascular fitness training, biofeedback, acupuncture and hypnotherapy, have shown limited efficacy (Berger et al. 2007). There is a need for new therapeutic options for the treatment of fibromyalgia and the promise of esreboxetine for clinical development cannot be ignored.

### SUMMARY OF THE INVENTION

Disclosed herein are salts of (2S)-2-[(S)-(ethoxyphenoxy)phenylmethyl]morpholine (esreboxetine) as shown in Formula I:

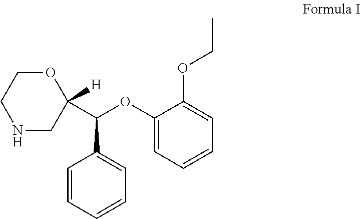 [SMILES: CCOc1ccccc1O[C@@H](c1ccccc1)[C@@H]1CNCCO1]

In some aspects the salt is an acid addition salt selected from adipic, L-ascorbic, L-aspartic, fumaric, glycolic, hydrochloric, maleic, mucic, phosphoric, sulfuric, and thiocyanic acid.

In some aspects, disclosed herein are pharmaceutical compositions comprising the acid addition salt of esreboxetine together with a pharmaceutically acceptable carrier, diluent or excipient.

In some aspects, the esrobxetine salts are crystalline.

In some aspects, the pharmaceutical compositions disclosed herein are used to treat conditions or disorders in which inhibition of norepinephrine uptake is indicated, such as, without limitation, fibromyalgia, ADHD, narcolepsy, obesity, depression, including unipolar depression, anxiety, cognitive function, panic disorders, bulimia nervosa, nocturnal enuresis, attenuate weight gain caused by atypical antipsychotics, such as olanzapine and chronic pain syndromes such as fibromyalgia and lower back pain.

All publications and patent applications mentioned in this specification are incorporated by reference in their entirety to the same extent as if each individual publication or patent application was specifically and individually indicated to be incorporated by reference.

### BRIEF DESCRIPTION OF THE DRAWINGS

FIG. 1 is an X-ray powder diffraction pattern (XRPD) overlay plot of adipate, fumarate, and glycolate crystalline salts of esreboxetine.

FIG. 2 is an XRPD overlay plot of aspartate, maleate and thiocyanate crystalline salts of esreboxetine.

FIG. 3 is an XRPD overlay plot of ascorbate, hydrochloride, and sulfate crystalline salts of esreboxetine.

FIG. 4 is an XRPD overlay plot of phosphate crystalline salt of esreboxetine.

FIG. 5 is an XRPD overlay plot of mucate crystalline salt of esreboxetine.

FIG. 6 is an XRPD overlay plot of esreboxetine fumarate forms A, B, and C.

FIG. 7 is an XRPD pattern of esreboxetine succinate.

FIG. 8 shows the asymmetric unit from the esreboxetine succinate crystal structure. Carbon atoms are gray, nitrogen atoms are gray with the letter N, oxygen atoms are black, and hydrogen atoms are white.

FIG. 9 shows a packing diagram from the esreboxetine succinate crystal structure looking down the a axis. Carbon atoms are are gray, nitrogen atoms are gray with the letter N, oxygen atoms are black. Hydrogen atoms are omitted for clarity.

FIG. 10 shows a packing diagram from esreboxetine succinate crystal structure looking down the b axis. Carbon atoms are gray, nitrogen atoms are gray with the letter N, and oxygen atoms are red. Hydrogen atoms are omitted for clarity.

FIG. 11 shows a packing diagram from esreboxetine succinate crystal structure looking down the c axis. Carbon atoms are gray, nitrogen atoms are gray with the letter N, and oxygen atoms are red. Hydrogen atoms are omitted for clarity.

FIG. 12 shows an overlay plot of XRPD pattern from sample esreboxetine succinate with a pattern calculated from single-crystal data.

FIG. 13 shows H¹ NMR for esreboxetine fumarate form A.

FIG. 14 shows H¹ NMR for esreboxetine fumarate form A.

FIG. 15 shows H¹ NMR for esreboxetine fumarate forms A+B.

FIG. 16 shows H¹ NMR for esreboxetine fumarate forms A+B.

FIG. 17 shows H¹ NMR for esreboxetine fumarate form B.

FIG. 18 shows H¹ NMR for esreboxetine fumarate form B.

FIG. 19 shows H¹ NMR for esreboxetine fumarate form C.

FIG. 20 shows H¹ NMR for esreboxetine fumarate form C.

### DETAILED DESCRIPTION OF THE EMBODIMENTS

Disclosed herein are salts of (2S)-2-[(S)-(ethoxyphenoxy)phenylmethyl]morpholine (e esreboxetine) as shown in Formula I:

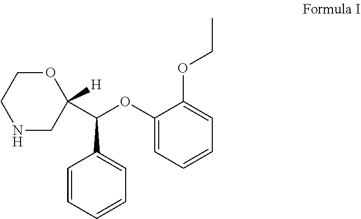 [SMILES: CCOc1ccccc1O[C@@H](c1ccccc1)[C@@H]1CNCCO1]

wherein an acid addition salt is selected from adipic, L-ascorbic, L-aspartic, fumaric, glycolic, hydrochloric, maleic, mucic, phosphoric, sulfuric, and thiocyanic acid.

In some embodiments, the salt is esreboxetine fumarate.

In some embodiments, the esreboxetine fumarate is crystalline.

In some embodiments, the salt is anhydrous crystalline esreboxetine fumarate Form A, B, C and/or a combination thereof.

In some embodiments, the salt is hydrated crystalline esreboxetine fumarate Form A, B, C and/or a combination thereof.

In another aspect, disclosed herein is a pharmaceutical composition comprising the salt esreboxetine fumarate with a pharmaceutically acceptable carrier, diluent or excipient.

In some embodiments, the salt of the pharmaceutical composition is anhydrous crystalline esreboxetine fumarate Form A, B, C and/or a combination thereof.

In some embodiments, the anhydrous esreboxetine fumarate crystalline Form A is characterized in that the crystalline form has an X-ray diffraction pattern (XRPD) comprising at least one peak at about 7.0 degrees 2θ.

As used herein, the meaning of the term “about” depends upon the context in which it is used. When used with respect to the position of a peak on an x-ray powder diffraction (XRPD) pattern, the term “about” includes peaks within ±0.1 degrees 2θ of the stated position. For example, as used herein, an XRPD peak at “about 10.0 degrees 2θ” means that the stated peak occurs from 9.9 to 10.1 degrees 2θ. When used with respect to the position of a peak on a solid state 13C NMR spectrum, the term “about” includes peaks within ±0.2 ppm of the stated position. For example, as used herein, a 13C NMR spectrum peak at “about 100.0 ppm” means that the stated peak occurs from 99.8 to 100.2 ppm.

In some embodiments, the anhydrous esreboxetine fumarate crystalline Form A exhibits an XRPD pattern at about 7.0 degrees 2θ and further comprises at least one peak selected from the group consisting of about 6.5 and 8.9 degrees 2θ.

In some embodiments, the anhydrous esreboxetine fumarate crystalline Form A exhibits an XRPD pattern at about 7 degrees 2θ and further comprises at least one peak selected from the group consisting of about 6.5, 8.9 12.5, 16.5, 17.9, 18.2, 21.0, and 24.0 degrees 2θ.

In some embodiments, the esreboxetine fumarate crystalline Form B is anhydrous.

In some embodiments, the anhydrous esreboxetine fumarate crystalline Form B is characterized in that the crystalline form has an X-ray diffraction pattern (XRPD) comprising at least one peak at about 5.9 degrees 2θ.

In some embodiments, the anhydrous esreboxetine fumarate crystalline Form B exhibits an XRPD pattern comprising at least one peak at about 5.9 degrees 2θ and further comprises at least one peak selected from the group consisting of about 11.5 and 17.2 degrees 2θ.

In some embodiments, the anhydrous esreboxetine fumarate crystalline form B exhibits an XRPD pattern comprising at least one peak at about 5.9 degrees 2θ and further comprises at least one peak selected from the group consisting of about 11.5, 17.2, 17.9, 20, and 23.2 degrees 2θ.

In some embodiments, the esreboxetine fumarate crystalline Form C is anhydrous.

In some embodiments, the anhydrous esreboxetine fumarate crystalline Form C is characterized in that the crystalline form has an X-ray diffraction pattern (XRPD) comprising at least one peak at about 6.5 degrees 2θ.

In some embodiments, the anhydrous esreboxetine fumarate crystalline Form C is characterized in that the crystalline form has an XRPD pattern comprising at least one peak at about 6.5 degrees 2θ and further comprising at least one peak selected from the group consisting of about 13 and 13.4 degrees 2θ.

In some embodiments, the anhydrous esreboxetine fumarate crystalline Form C exhibits an XRPD pattern comprising at least one peak at about 6.5 degrees 2θ and further comprising at least one peak selected from the group consisting of about 13, 13.4, 14.8, 15.2, 18, 18.5, 19.2, 20, 21, 22.4, and 23.5 degrees 2θ.

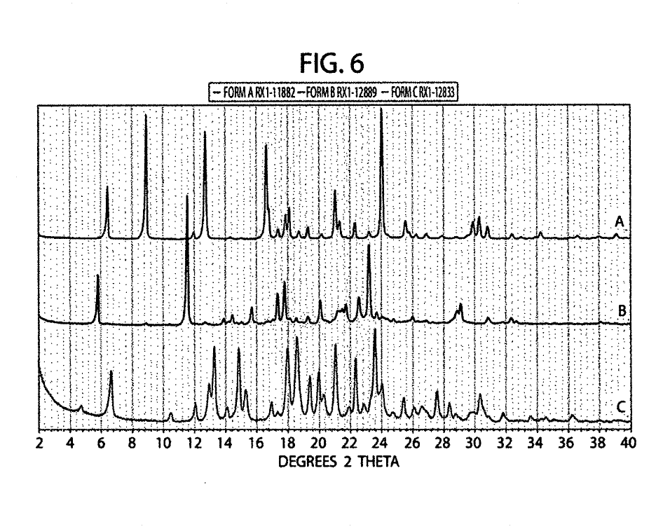

In some embodiments, the anhydrous esreboxetine fumarate crystalline Form A exhibits an XRPD pattern substantially the same as FIG. 6.

In some embodiments, the anhydrous esreboxetine fumarate crystalline Form B exhibits an XRPD pattern substantially the same as FIG. 6.

In some embodiments, the anhydrous esreboxetine fumarate crystalline Form C exhibits an XRPD pattern substantially the same as FIG. 6.

In some embodiments, the anhydrous esreboxetine fumarate crystalline Form A is characterized by at least one of:

a. an XPRD pattern exhibiting at least four of the peaks shown in FIG. 6; and

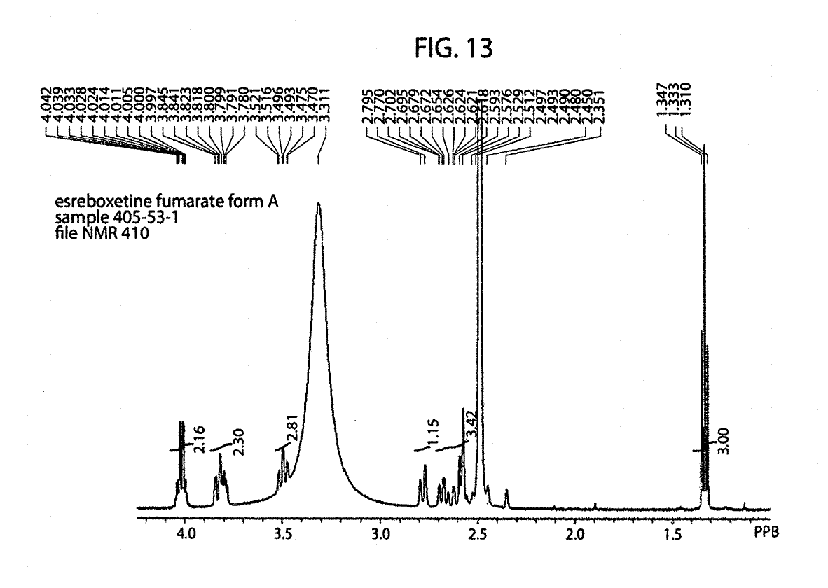

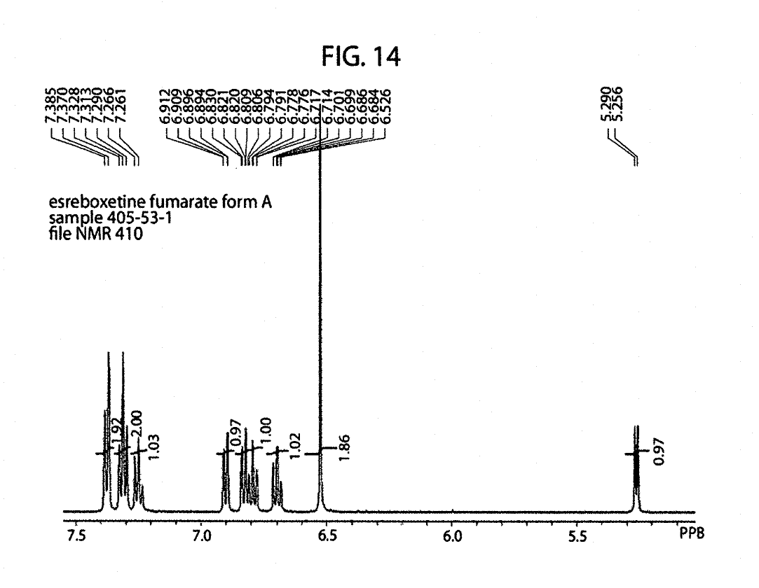

b. an NMR spectrum substantially the same as FIGS. 13 and 14.

In some embodiments, the anhydrous esreboxetine fumarate crystalline Form B is characterized by at least one of:

a. an XPRD pattern exhibiting at least four of the peaks shown in FIG. 6; and

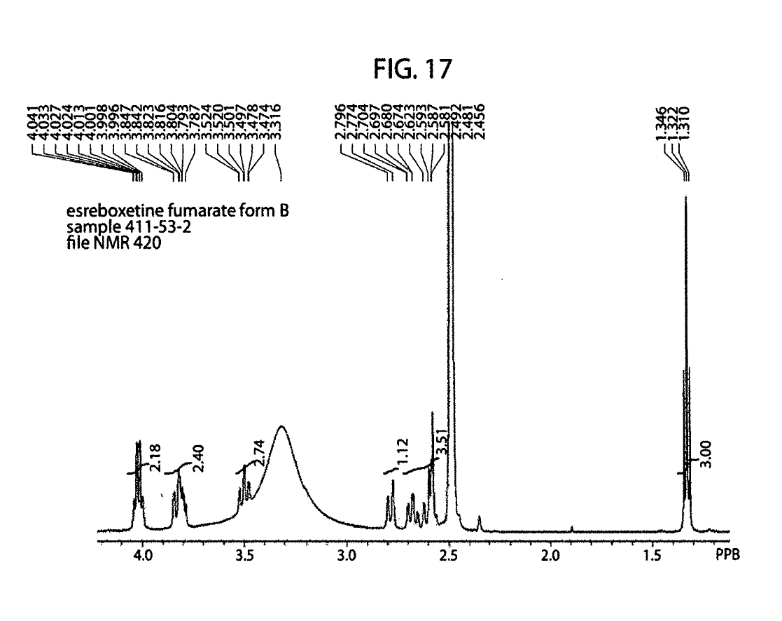

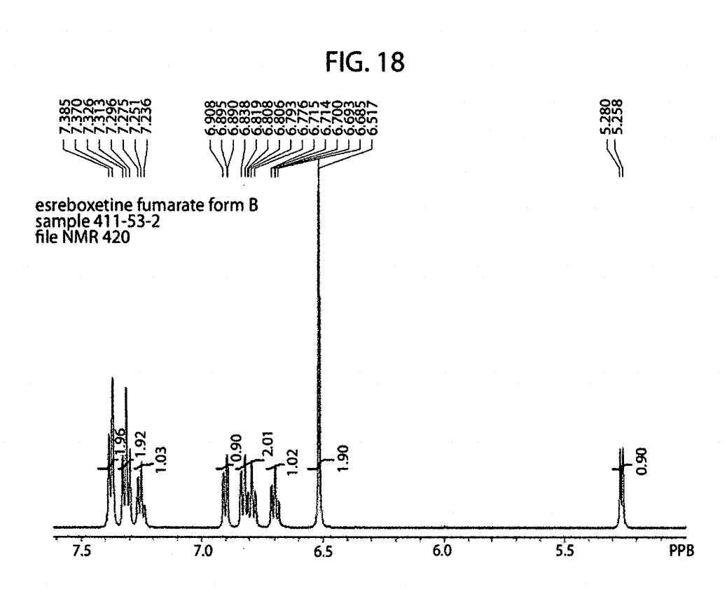

b. an NMR spectrum substantially the same as FIGS. 17 and 18.

In some embodiments, the anhydrous esreboxetine fumarate crystalline Form C is characterized by at least one of:

a. an XPRD pattern exhibiting at least four of the peaks shown in FIG. 6; and

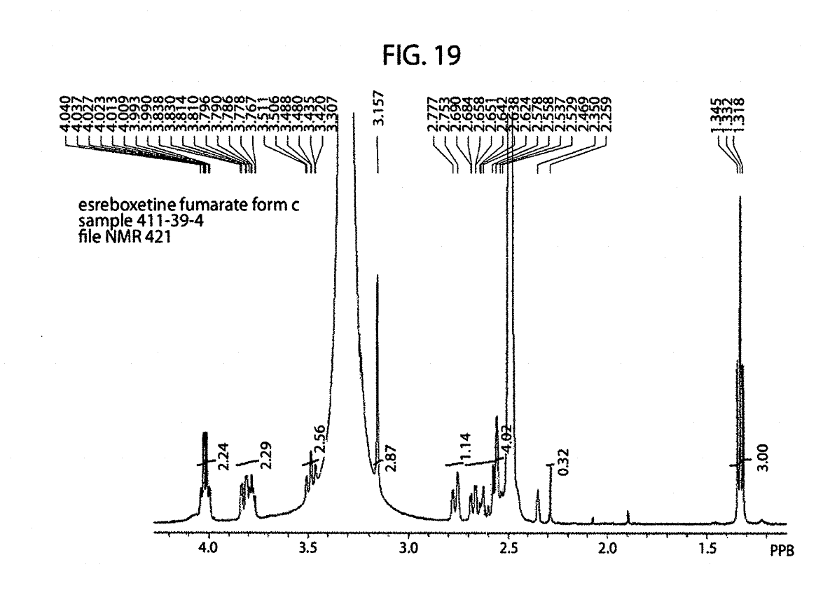

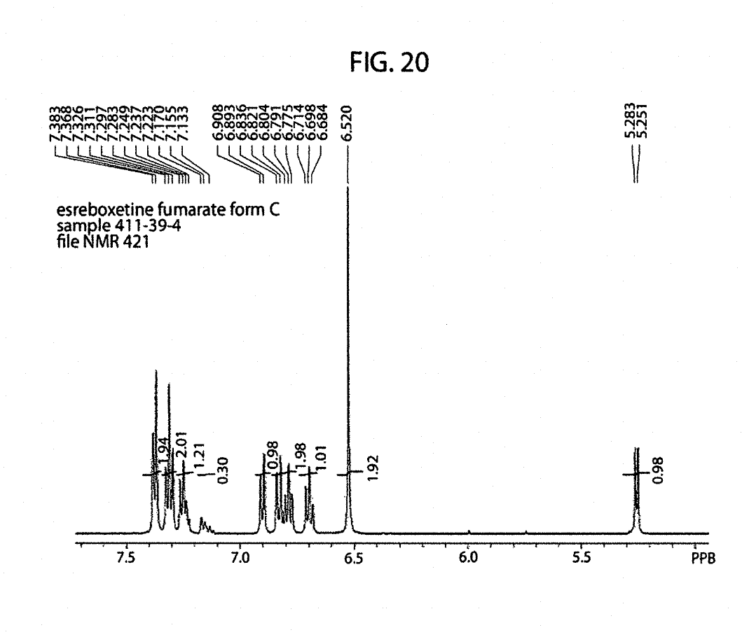

b. an NMR spectrum substantially the same as FIGS. 19 and 20.

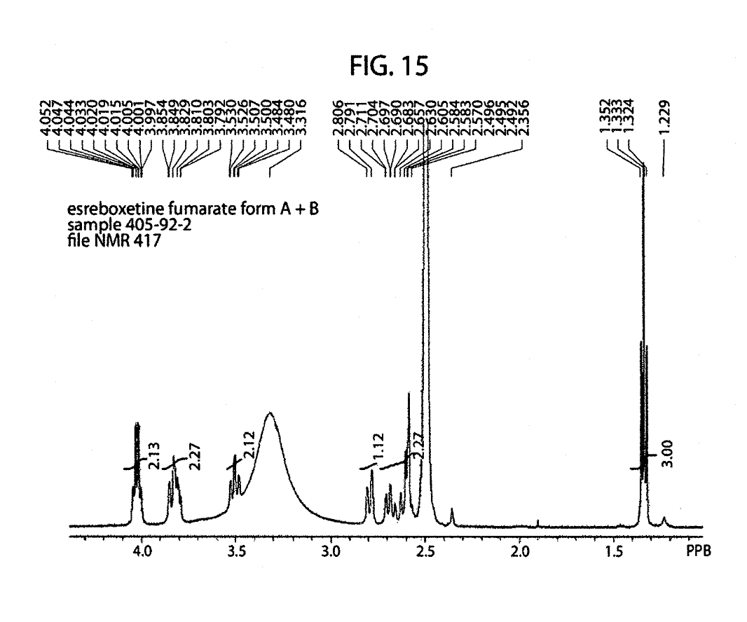

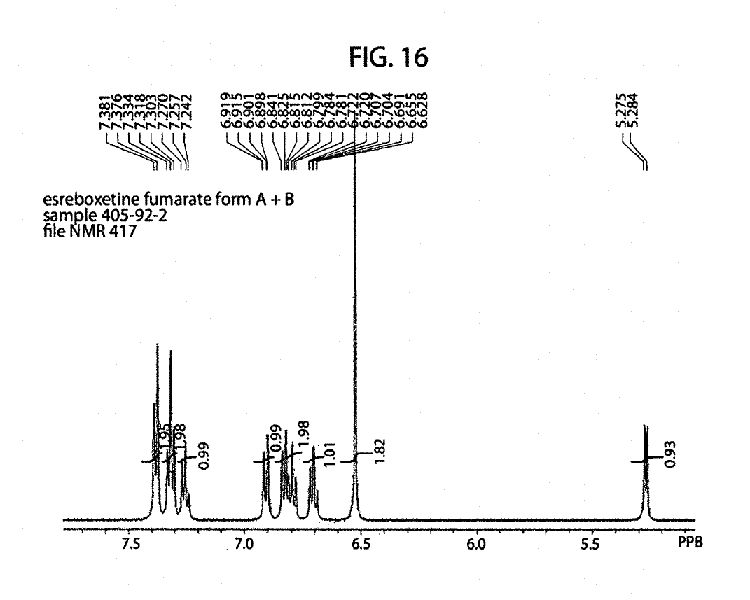

In some embodiments, the anhydrous esreboxetine fumarate crystalline Forms A+B are characterized by an NMR spectrum substantially the same as FIGS. 15 and 16.

In some embodiments, the salt is esreboxetine adipate.

In some embodiments, the esreboxetine adipate is crystalline.

In some embodiments, the salt is anhydrous crystalline esreboxetine adipate Form A.

In another aspect, disclosed herein is a pharmaceutical composition comprising the salt esreboxetine adipate with a pharmaceutically acceptable carrier, diluent or excipient.

In some embodiments, the salt of the pharmaceutical composition is anhydrous crystalline esreboxetine adipate Form A.

In some embodiments, the anhydrous esreboxetine adipate crystalline Form A is characterized in that the crystalline form has an X-ray diffraction pattern (XRPD) comprising at least one peak at about 7.2 degrees 2θ.

In some embodiments, the anhydrous esreboxetine adipate crystalline Form A exhibits an XRPD pattern comprising at least one peak at about 7.2 degrees 2θ and further comprising at least one peak selected from the group consisting of about 13.9 and 20.9 degrees 2θ.

In some embodiments, the anhydrous esreboxetine adipate crystalline Form A exhibits an XRPD pattern comprises at least one peak at about 7.2 degrees 2θ and further comprises at least one peak selected from the group consisting of about 13.9, 20.9, 21.9, 22.4, 23.5, and 24.2 degrees 2θ.

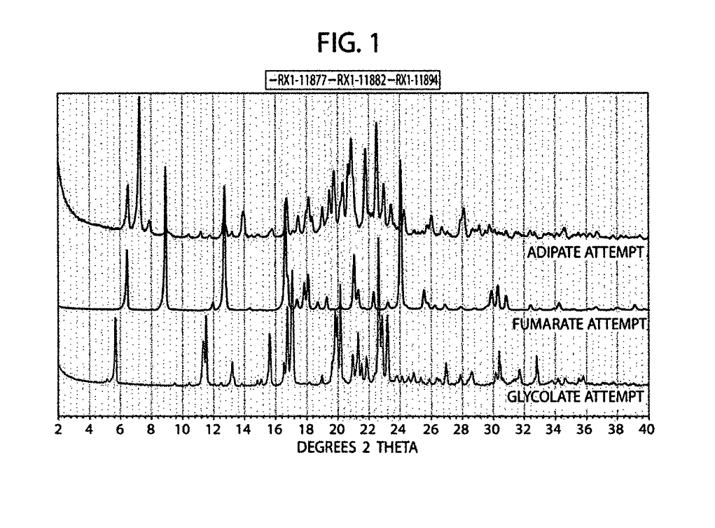

In some embodiments, the anhydrous esreboxetine adipate crystalline Form exhibits an XRPD pattern substantially the same as FIG. 1.

In some embodiments, the salt is esreboxetine glycolate.

In some embodiments, the esreboxetine glycolate is crystalline.

In some embodiments, the salt is anhydrous crystalline esreboxetine glycolate Form A.

In another aspect, disclosed herein is a pharmaceutical composition comprising the salt esreboxetine glycolate with a pharmaceutically acceptable carrier, diluent or excipient.

In some embodiments, the salt of the pharmaceutical composition is anhydrous crystalline esreboxetine glycolate Form A.

In some embodiments, the anhydrous esreboxetine glycolate crystalline Form A is characterized in that the crystalline form has an X-ray diffraction pattern (XRPD) comprising at least one peak at about 5.8 degrees 2θ.

In some embodiments, the anhydrous esreboxetine glycolate crystalline Form A exhibits an XRPD pattern comprising at least one peak at about 5.8 and further comprising at least one peak selected from the group consisting of about 11.2 degrees 2θ.

In some embodiments, the anhydrous esreboxetine glycolate crystalline Form A exhibits an XRPD pattern comprising at least one peak at about 5.8 and further comprising at least one peak selected from the group consisting of about 11.2, 11.4, 13.2, 15.8, 16.8, 17, 19.8, 19.9, and 20.2 degrees 2θ.

In some embodiments, the anhydrous esreboxetine glycolate crystalline Form exhibits an XRPD pattern substantially the same as FIG. 1.

In some embodiments, the salt is esreboxetine aspartate.

In some embodiments, the esreboxetine aspartate is crystalline.

In some embodiments, the salt is anhydrous crystalline esreboxetine aspartate Form A.

In another aspect, disclosed herein is a pharmaceutical composition comprising the salt esreboxetine aspartate with a pharmaceutically acceptable carrier, diluent or excipient.

In some embodiments, the salt of the pharmaceutical composition is anhydrous crystalline esreboxetine aspartate Form A.

In some embodiments, the anhydrous esreboxetine aspartate crystalline Form A is characterized in that the crystalline form has an X-ray diffraction pattern (XRPD) comprising at least one peak at about 8.5 degrees 2θ.

In some embodiments, the anhydrous esreboxetine aspartate crystalline Form A exhibits an XRPD pattern comprising at least one peak at about 8.5 and further comprising at least one peak selected from the group consisting of about 12.0 and 13.2 degrees 2θ.

In some embodiments, the anhydrous esreboxetine aspartate crystalline Form A exhibits an XRPD pattern comprising at least one peak at about 8.5 and further comprising at least one peak selected from the group consisting of about 12, 13.2, 14.4, 14.9, 15, 17.6, 20.1, 21.1, and 22.0 degrees 2θ.

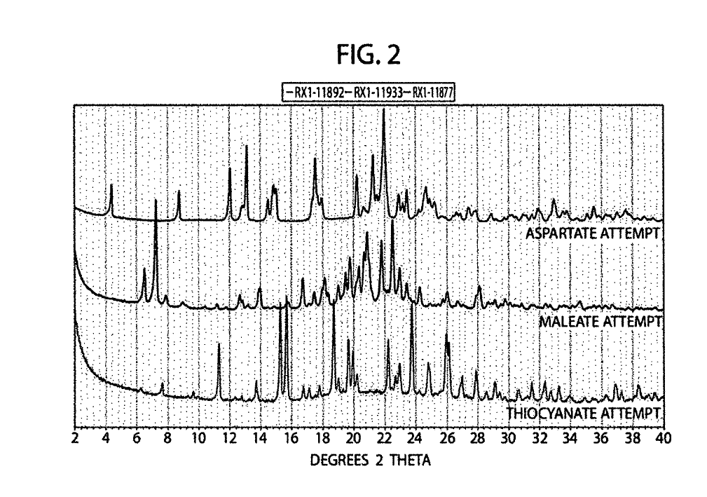

In some embodiments, the anhydrous esreboxetine aspartate crystalline Form exhibits an XRPD pattern substantially the same as FIG. 2.

In some embodiments, the salt is esreboxetine maleate.

In some embodiments, the esreboxetine maleate is crystalline.

In some embodiments, the salt is anhydrous crystalline esreboxetine maleate Form A.

In another aspect, disclosed herein is a pharmaceutical composition comprising the salt esreboxetine maleate with a pharmaceutically acceptable carrier, diluent or excipient.

In some embodiments, the salt of the pharmaceutical composition is anhydrous crystalline esreboxetine maleate Form A.

In some embodiments, the anhydrous esreboxetine maleate crystalline Form A is characterized in that the crystalline form has an X-ray diffraction pattern (XRPD) comprising at least one peak at about 6.4 degrees 2θ.

In some embodiments, the anhydrous esreboxetine maleate crystalline Form A exhibits an XRPD pattern comprising at least one peak at about 6.4 and further comprising at least one peak selected from the group consisting of about 7.0 and 14.0 degrees 2θ.

In some embodiments, the anhydrous esreboxetine maleate crystalline Form A exhibits an XRPD pattern comprising at least one peak at about 6.4 and further comprising at least one peak selected from the group consisting of about 7, 14, 16.8, 17.9, 20.4, 21, 21.8, and 22.8 degrees 2θ.

In some embodiments, the anhydrous esreboxetine maleate crystalline Form exhibits an XRPD pattern substantially the same as FIG. 2.

In some embodiments, the salt is esreboxetine thiocyanate.

In some embodiments, the esreboxetine thiocyanate is crystalline.

In some embodiments, the salt is anhydrous crystalline esreboxetine thiocyanate.

In another aspect, disclosed herein pharmaceutical composition comprising the salt esreboxetine thiocyanate with a pharmaceutically acceptable carrier, diluent or excipient.

In some embodiments, the salt of the pharmaceutical composition is anhydrous crystalline esreboxetine thiocyanate.

In some embodiments, the anhydrous esreboxetine thiocyanate crystalline Form A is characterized in that the crystalline form has an X-ray diffraction pattern (XRPD) comprising at least one peak at about 11.4 degrees 2θ.

In some embodiments, the anhydrous esreboxetine thiocyanate crystalline Form A exhibits an XRPD pattern comprising at least one peak at about 11.4 and further comprising at least one peak selected from the group consisting of about 15.2 and 15.8 degrees 2θ.

In some embodiments, the anhydrous esreboxetine thiocyanate crystalline Form A exhibits an XRPD pattern comprising at least one peak at about 11.4 and further comprising at least one peak selected from the group consisting of about 15.2, 15.8, 18.9, 19.7, 20, 22.2, 23, and 22.9 degrees 2θ.

In some embodiments, the anhydrous esreboxetine thiocyanate crystalline Form A exhibits an XRPD pattern substantially the same as FIG. 2.

In some embodiments, the salt is esreboxetine ascorbate.

In some embodiments, the esreboxetine ascorbate is crystalline.

In some embodiments, the salt is anhydrous crystalline esreboxetine ascorbate Form A.

In another aspect, disclosed herein is a pharmaceutical composition comprising the salt esreboxetine ascorbate with a pharmaceutically acceptable carrier, diluent or excipient.

In some embodiments, the salt of the pharmaceutical composition is anhydrous crystalline esreboxetine ascorbate.

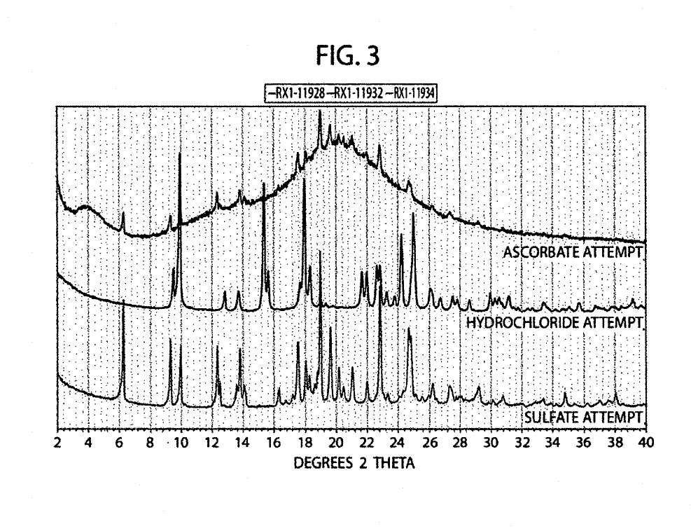

In some embodiments, the crystalline esreboxetine ascorbate exhibits an XRPD pattern substantially the same as FIG. 3.

In some embodiments, the salt is esreboxetine hydrochloride.

In some embodiments, the esreboxetine hydrochloride is crystalline.

In some embodiments, the salt is anhydrous crystalline esreboxetine hydrochloride.

In another aspect, disclosed herein is a pharmaceutical composition comprising the salt esreboxetine hydrochloride with a pharmaceutically acceptable carrier, diluent or excipient.

In some embodiments, the salt of the pharmaceutical composition is anhydrous crystalline esreboxetine hydrochloride.

In some embodiments, the anhydrous esreboxetine hydrochloride crystalline form is characterized in that the crystalline form has an X-ray diffraction pattern (XRPD) comprising at least one peak at about 9.9 degrees 2θ.

In some embodiments, the anhydrous esreboxetine hydrochloride crystalline form exhibits an XRPD pattern comprising at least one peak at about 9.9 and further comprising at least one peak selected from the group consisting of about 12.9 and 13.8 degrees 2θ.

In some embodiments, the anhydrous esreboxetine hydrochloride crystalline form exhibits an XRPD pattern comprising at least one peak at about 9.9 and further comprising at least one peak selected from the group consisting of about 12.9, 13.8, 15.4, 15.7, 18, 18.4, 21.7, and 22 degrees 2θ.

In some embodiments, the anhydrous esreboxetine hydrochloride crystalline form exhibits an XRPD pattern substantially the same as FIG. 3.

In some embodiments, the salt is Esreboxetine sulfate.

In some embodiments, the Esreboxetine sulfate is crystalline.

In some embodiments, the salt is anhydrous crystalline Esreboxetine sulfate.

In another aspect, disclosed herein is a pharmaceutical composition comprising the salt Esreboxetine sulfate with a pharmaceutically acceptable carrier, diluent or excipient.

In some embodiments, the salt of the pharmaceutical composition is anhydrous crystalline Esreboxetine sulfate.

In some embodiments, the anhydrous Esreboxetine sulfate crystalline form is characterized in that the crystalline form has an X-ray diffraction pattern (XRPD) comprising at least one peak at about 6.2 degrees 2θ.

In some embodiments, the anhydrous Esreboxetine sulfate crystalline form exhibits an XRPD pattern further comprising at least one peak selected from the group consisting of about 9.2 and 10.0 degrees 2θ.

In some embodiments, the anhydrous Esreboxetine sulfate crystalline form exhibits an XRPD pattern further comprising at least one peak selected from the group consisting of about 12.3, 13.9, 17.4, 19.2, 19.8, and 22.8 degrees 2θ.

In some embodiments, the anhydrous esreboxetine sulfate crystalline form exhibits an XRPD pattern substantially the same as FIG. 3.

In some embodiments, the salt is esreboxetine phosphate.

In some embodiments, the esreboxetine phosphate is crystalline.

In some embodiments, the salt is anhydrous crystalline esreboxetine phosphate.

In another aspect, disclosed herein is a pharmaceutical composition comprising the salt esreboxetine phosphate with a pharmaceutically acceptable carrier, diluent or excipient.

In some embodiments, the salt of the pharmaceutical composition is anhydrous crystalline esreboxetine phosphate.

In some embodiments, the anhydrous esreboxetine phosphate crystalline is characterized in that the crystalline form has an X-ray diffraction pattern (XRPD) comprising at least one peak at about 15.9 degrees 2θ.

In some embodiments, the anhydrous esreboxetine phosphate crystalline form exhibits an XRPD pattern comprising at least one peak at about 15.9 and further comprising at least one peak selected from the group consisting of about 17.9 and 21.4 degrees 2θ.

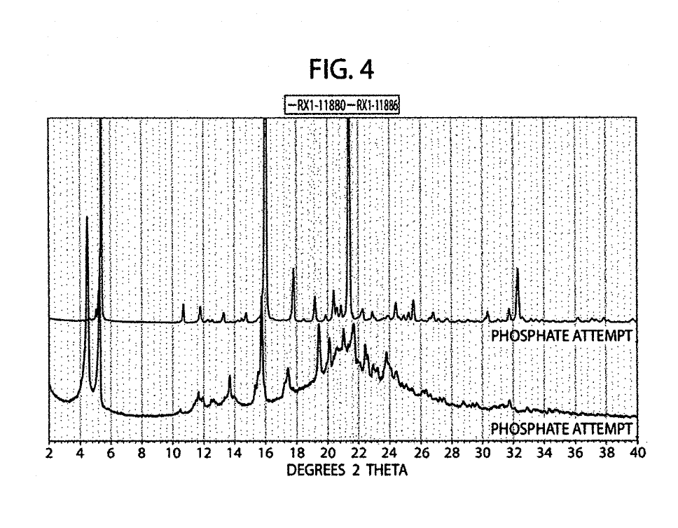

In some embodiments, the anhydrous esreboxetine phosphate crystalline form exhibits an XRPD pattern substantially the same as FIG. 4.

In some embodiments, the salt is esreboxetine mucate.

In some embodiments, the esreboxetine mucate is crystalline.

In some embodiments, the salt is anhydrous crystalline esreboxetine mucate.

In another aspect, disclosed herein is a pharmaceutical composition comprising the salt esreboxetine mucate with a pharmaceutically acceptable carrier, diluent or excipient.

In some embodiments, the salt of the pharmaceutical composition is anhydrous crystalline esreboxetine mucate.

In some embodiments, the anhydrous esreboxetine mucate crystalline is characterized in that the crystalline form has an X-ray diffraction pattern (XRPD) comprising at least one peak at about 15.9 degrees 2θ.

In some embodiments, the anhydrous esreboxetine mucate crystalline form exhibits an XRPD pattern comprising at least one peak at about 15.9 and further comprising at least one peak selected from the group consisting of about 16.0 and 16.9 degrees 2θ.

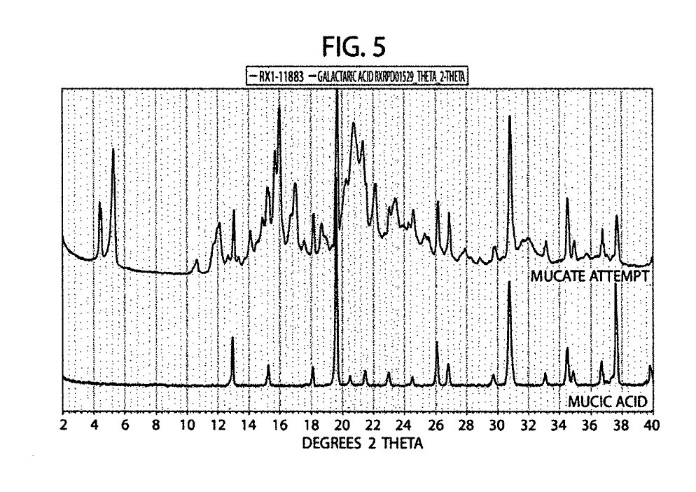

In some embodiments, the anhydrous esreboxetine mucate crystalline form exhibits an XRPD pattern substantially the same as FIG. 5.

Various crystalline salts of esreboxetine were generated with the potential of not altering the efficacy or safety profile that esreboxetine has demonstrated in the treatment of fibromyalgia. For one particular salt, esreboxetine fumarate, three different polymorphs were generated and characterized. The melting point of esreboxetine fumarate was 170.27° C., much higher than the melting point for esreboxetine succinate which is 147.67° C. Compounds with higher melting points are more amenable to tablet formation due to their increased crystalline stability and improved product performance, especially with regards to shelf life and compatibility with other ingredients used in tablet formation.

Disclosed herein are salts and polymorph forms of esreboxetine or (2S)-2-[(S)-(2-ethoxyphenoxy)-phenylmethyl]morpholine. The salts and polymorphs of esreboxetine (2S)-2-[(S)-(2-ethoxyphenoxy)-phenylmethyl]morpholine disclosed herein are based on solvates, hydrates or conjugates of esreboxetine or (2S)-2-[(S)-(2-ethoxyphenoxy)-phenylmethyl]morpholine. The solvates are formed by combining esreboxetine with one or more pharmaceutically acceptable salts noncovalently. The term “pharmaceutically acceptable salt” refers to salts which retain the biological effectiveness and properties of the compounds disclosed herein and which are not biologically or otherwise undesirable. In some cases, the compounds disclosed herein are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diary) amines, triaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heterocyclic, and the like. Also included are amines where the two or three substituents, together with the amino nitrogen, form a heterocyclic group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include, without limitation, hydrochloric acid, hydrobromic acid, sulfuric acid, nitric acid, phosphoric acid, and the like. Salts derived from organic acids include, without limitation, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Hydrates are formed by combining esreboxetine with water noncovalently. Hydrates may include monohydrates, dihydrates, trihydrates, tetrahydrates, and so on. Conjugates are formed by combining covalently esreboxetine and a conjugateable chemical. A preferred conjugatable chemical is polyethylene glycol of between 100 to 10000 molecular weight.

The methods disclosed herein can be used to administer esreboxetine to patients to treat any disorder that is now known or that is later discovered to be treatable with such compounds particularly compounds that are norepinephrine uptake inhibitors.

Suitable routes of administration include, but are not limited to, inhalation, transdermal, oral, rectal, transmucosal, intestinal and parenteral administration, including intramuscular, subcutaneous and intravenous injections. For any mode of administration, the actual amount of esreboxetine delivered, as well as the dosing schedule necessary to achieve the advantageous pharmacokinetic profiles described herein, will be depend, in part, on such factors as the bioavailability of esreboxetine, the disorder being treated, the desired therapeutic dose, and other factors that will be apparent to those of skill in the art. The actual amount delivered and dosing schedule can be readily determined by those of skill without undue experimentation by monitoring the blood plasma levels of administered drug, and adjusting the dosage or dosing schedule as necessary to achieve the desired pharmacokinetic profile.

Esreboxetine, or pharmaceutically acceptable salts and/or hydrates thereof, may be administered singly, in combination with other compounds, and/or in combination with other therapeutic agents, including cancer chemotherapeutic agents. Esreboxetine may be administered alone or in the form of a pharmaceutical composition, wherein the drug is in admixture with one or more pharmaceutically acceptable carriers, excipients or diluents. Pharmaceutical compositions for use in accordance with the present disclosure may be formulated in conventional manner using one or more physiologically acceptable carriers comprising excipients and auxiliaries which facilitate processing of the drug into preparations which can be used pharmaceutically. Proper formulation is dependent upon the route of administration chosen. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, esreboxetine can be formulated readily by combining drug with pharmaceutically acceptable carriers well known in the art. Such carriers enable esreboxetine to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of esreboxetine doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, esreboxetine for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Esreboxetine may be formulated for parenteral administration by injection, e.g., by bolus injection or continuous infusion. It is preferred that esreboxetine be administered by continuous infusion subcutaneously over a period of 15 minutes to 24 hours. Formulations for injection may be presented in unit dosage form, e.g., in ampoules or in multi-dose containers, with an added preservative. Esreboxetine may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of esreboxetine in water-soluble form. Additionally, suspensions of esreboxetine may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

Esreboxetine may also be formulated in rectal compositions such as suppositories or retention enemas, e.g., containing conventional suppository bases such as cocoa butter or other glycerides.

In addition to the formulations described previously, esreboxetine may also be formulated as a depot preparation. Such long acting formulations may be administered by implantation (for example subcutaneously or intramuscularly) or by intramuscular injection.

Thus, for example, esreboxetine may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use with the present invention include compositions wherein the active ingredient is contained in a therapeutically effective amount, i.e., an amount effective to achieve its intended purpose. Of course, the actual amount of active ingredient will depend on, among other things, its intended purpose. Determination of an effective amount is well within the capabilities of those skilled in the art, especially in light of the detailed disclosure herein.

For other modes of administration, dosage amount and interval can be adjusted individually to provide effective plasma and/or tissue levels of the administered compound, and/or a metabolite thereof, according to the pharmacokinetic profiles described herein, as previously described.

The actual amount of composition administered will, of course, be dependent on the subject being treated, the subject's weight, the severity of the affliction, the mode of administration and the judgment of the prescribing physician.

The disclosure will now be described with reference to the following examples which illustrate some particular aspects and embodiments of the present application. However, it is to be understood that the particularity of the following description is not to supersede the generality of the preceding detailed description and/or summary of the aspects and embodiments of the disclosure.

### EXAMPLES

### Salt Screening

To begin the process of identifying new salt and polymorph compositions of esreboxetine, a salt screen of esreboxetine was carried out. The acids used in those experiments were adipic, L-ascorbic, L-aspartic, fumaric, glycolic, hydrochloric, maleic, mucic, phosphoric, sulfuric, and thiocyanic acid. The fumarate salt was made at larger scale and characterized.

Esreboxetine was mixed with various acids under various conditions in attempts to generate crystalline salts (see Tables 3-5).

TABLE 3
Samples Generated and Analyzed

| Acid | Conditions^{a} | XRPD Pattern^{b} |
|---|---|---|
| acetic | E, MeOH, RT; gel. Vac. desic; | — |
| | gel. Et₂O triturate; gel. Vac. desic; | |
| | gel | |
| | C, acetone, RT → −15° C.; E; gel. | — |
| | Vac. desic.; gel. Et₂O triturate; | |
| | gel. Vac. desic.; gel | |
| | C, EtOH/hex, RT → −15° C.; E; | — |
| | gel.. Vac. desic.; gel. Et₂O | |
| | triturate; gel. Vac. desic.; gel | |
| adipic | E, MeOH, RT; gel. Vacuum | NC |
| | desiccator | |
| | C, acetone, RT → −15° C. | New |
| | C, EtOH/hex, RT → −15° C. | NC |
| L-ascorbic | E, MeOH, RT; gel. Vacuum | NC |
| | desiccator | |
| | C, acetone, RT → −15° C.; E; gel. | new + NC |
| | Vacuum desiccator | |
| | C, EtOH/hex, RT → −15° C. | NC |
| L-aspartic | SL, 95:5 ACN/water, 80° C., 5 | new + NC |
| | days; gel. Vac. desic; gel. Et₂O | |
| | triturate | |
| | Grind, water, ~20 mins | New |
| benzoic | E, MeOH, RT; gel. Vacuum | NC |
| | desiccator | |
| | C, acetone, RT → −15° C.; E; gel. | NC |
| | Vacuum desiccator | |
| | C, EtOH/hex, RT → −15° C.; E; | NC |
| | gel. Vacuum desiccator | |
| citric | E, MeOH, RT; gel. Vacuum | NC |
| | desiccator | |
| | C, acetone, RT → −15° C.; E; gel. | NC |
| | Vacuum desiccator | |
| | C, EtOH/hex, RT → −15° C.; E; | NC |
| | gel. Vacuum desiccator | |
| cyclamic | E, MeOH, RT; gel. Vacuum | NC |
| | desiccator | |
| | C, acetone, RT → −15° C.; E; gel. | NC |
| | Vacuum desiccator | |
| | C, EtOH/hex, RT → −15° C.; E; | NC |
| | gel. Vacuum desiccator | |

^{a}C = cool, EtOH = absolute ethanol; Et₂O = ethyl ether, E = evaporation, hex = hexanes, M = molar, MeOH = methanol, P = precipitation, RT = room temperature, SL = slurry
^{b}NC = non-crystalline

TABLE 4
Samples Generated and Analyzed

| Acid | Conditions^{a} | XRPD Pattern^{b} |
|---|---|---|
| fumaric | E, MeOH, RT; gel. Vacuum | new |
| | desiccator | |
| | C, acetone, RT → −15° C. | |
| | C, EtOH/hex, RT | |
| galactaric (mucic) | E, MeOH, RT; gel. Vacuum | new + acid |
| | desiccator | |
| | C, acetone, RT → −15° C.; E; gel. | NC + acid |
| | Vacuum desiccator | |
| | C, EtOH/hex, RT → −15° C. | Acid |
| glutaric | E, MeOH, RT; gel. Vacuum | NC |
| | desiccator | |
| | C, acetone, RT → −15° C.; E; gel. | NC |
| | Vacuum desiccator | |
| | C, EtOH/hex, RT → −15° C.; E; | NC |
| | gel. Vacuum desiccator | |
| glycolic | E, MeOH, RT; gel. Vacuum | new |
| | desiccator | |
| | C, acetone, RT → −15° C.; E; gel. | |
| | Vacuum desiccator | |
| | C, EtOH/hex, RT → −15° C.; E | |
| hippuric | E, MeOH, RT; gel. Vacuum | NC |
| | desiccator | |
| | C, acetone, RT → −15° C.; E; gel. | NC |
| | Vacuum desiccator | |
| | C, EtOH/hex, RT → −15° C.; E; | NC |
| | gel. Vacuum desiccator | |
| hydrochloric | E, MeOH, RT; gel. Vacuum | NC |
| | desiccator | |
| | C, acetone, RT → −15° C.; E; gel. | new |
| | Vacuum desiccator | |
| | C, EtOH/hex, RT → −15° C.; E; | |
| | gel. Vacuum desiccator | |
| maleic | E, MeOH, RT; gel. Vacuum | new |
| | desiccator | |
| | C, acetone, RT → −15° C.; E; gel. | |
| | Vacuum desiccator | |
| | C, EtOH/hex, RT → −15° C.; E; | NC |
| | gel. Vacuum desiccator | |

^{a}C = cool, EtOH = absolute ethanol; Et₂O = ethyl ether, E = evaporation, hex = hexanes, M = molar, MeOH = methanol, P = precipitation, RT = room temperature, SL = slurry
^{b}NC = non-crystalline

TABLE 5
Samples Generated and Analyzed

| Acid | Conditions^{a} | XRPD Pattern^{b} |
|---|---|---|
| L-malic | E, MeOH, RT; gel. Vacuum | NC |
| | desiccator | |
| | C, acetone, RT → −15° C. | NC |
| | C, EtOH/hex, RT → −15° C.; E; | NC |
| | gel. Vacuum desiccator | |
| phosphoric | E, MeOH, RT; gel. Vacuum | new 1 |
| | desiccator | |
| | SL, acetone, RT | new 2 + NC |
| | C, EtOH/hex, RT → −15° C. | |
| sebacic | E, MeOH, RT; gel. Vacuum | NC |
| | desiccator | |
| | C, acetone, RT → −15° C. | NC |
| | C, EtOH/hex, RT → −15° C.; E; | NC |
| | gel. Vacuum desiccator | |
| sulfuric | E, MeOH, RT; gel. Vacuum | NC |
| | desiccator | |
| | C, acetone, RT → −15° C.; E; gel. | new |
| | Vacuum desiccator | |
| | C, EtOH/hex, RT → −15° C. | NC |
| L-tartaric | E, MeOH, RT; gel. Vacuum | NC |
| | desiccator | |
| | C, acetone, RT → −15° C. | NC + pks |
| | C, EtOH/hex, RT → −15° C. | NC + pks |
| thiocyanic | E, MeOH, RT; gel. Vacuum | new |
| | desiccator | |
| | C, acetone, RT → −15° C. | |
| | C, EtOH/hex, RT → −15° C. | |

^{a}C = cool, EtOH = absolute ethanol; Et₂O = ethyl ether, E = evaporation, hex = hexanes, M = molar, MeOH = methanol, P = precipitation, RT = room temperature, SL = slurry
^{b}NC = non-crystalline

Eleven samples were found that exhibited an XRPD pattern suggestive of new phase formation. That is, the patterns contain peaks that do not arise from either free base esreboxetine or the acid used (see Tables 3-5). Overlay plots of the XRPD patterns are shown in FIGS. 1 through 5.

All samples having an XRPD pattern suggestive of new phase formation were analyzed by DSC and TG. The results are summarized in Table 6. Samples that were mixtures (containing unreacted acid) or that were poorly crystalline were not analyzed.

TABLE 6
Thermal Analysis of Crystalline Salts

| Salt | Results |
|---|---|
| adipate | endo 96.65, 210.89° C. |
| | 1.42% start to 100° C. |
| | 44.02% 100 to 240° C. |
| aspartate | endo 68.70° C. |
| | 9.68% start to 100° C. |
| fumarate | endo 170.27° C. |
| | 1.11% start to 170° C. |
| glycolate | endo 74.97° C. |
| | 4.25% start to 100 |
| hydrochloride | endo 73.75, 131.90° C. |
| | 1.90% start to 150 |
| maleate | endo 73.75, 125.95° C. |
| | 0.59% start to 135° C. |
| phosphate | endo 58.45, 95.42° C. |
| | 4.47% start to 100° C. |
| sulfate | endo 72.26 |
| | 3.14 start to 80° C. |
| thiocyanate | endo 118.14° C. |
| | 2.00% start to 125° C. |

After noting the endothermic event at 170.27° C., for esreboxetine fumarate, a temperature much higher than that of the other salts, the fumarate salt was thus chosen to prepare at larger scale for further characterization (Table 7).

TABLE 7
Preparation of Salts at Larger Scale

| | | | | XRPD |
|---|---|---|---|---|
| Salt | Method | Solvent | Conditions | Pattern |
| fumarate | cool | acetone | −15° C., 3 days | new |

### Characterization of the Salts

Fumarate Salt:

Characterization data are shown in Table 8. It is a 1:1 API:acid salt (approximately 1 mole of fumaric acid is observed in the NMR spectrum). It is unsolvated; TG results show 0.83% weight loss below 165° C. The endothermic event observed by DSC at 165.04° C. is likely melting. It is slightly hygroscopic.

With an observed melting point approximately 20° C. higher than that of esreboxetine succinate, esreboxetine fumarate potentially provides better stability with regards to shelf life and compatibility with other ingredients of tablet formulations. In addition, the increased stability of compounds with higher melting points renders the tablet product more susceptible to dissolution and disintegration; properties ideally suited for immediate release oral formulations.

TABLE 8
Characterization Data for Esreboxetine Fumarate Salt
(sample 405-53-1)

| Technique | Result |
|---|---|
| XRPD | crystalline, fumarate A |
| DSC | endo 165.04° C. |
| TG | 0.83% start to 165° C. |
| DVS | 0.24% loss upon drying at 5% RH 1.80% gain from 5 |
| | to 95% RH |
| | 1.97% loss from 95 to 5% RH |
| Post-DVS XRPD | unchanged |
| NMR | consistent with a 1:1 (API:acid) salt |

Conclusions from the Salt Screen

A salt screen of esreboxetine was carried out. Eleven samples were found that exhibit an XRPD pattern suggestive of new phase formation. That is, the patterns contain peaks that do not arise from either esreboxetine or the acid used. The acids used in those experiments were adipic, L-ascorbic, L-aspartic, fumaric, glycolic, hydrochloric, maleic, mucic, phosphoric, sulfuric, and thiocyanic acid. The fumarate salt was made at larger scale and characterized due to the higher melting point that was observed for all of the other salts.

### Esreboxetine Fumarate Polymorph Screening

Esreboxetine fumarate was mixed with various solvents under various conditions in attempts to generate polymorphs. Samples generated and analyzed are listed in Tables 9-11.

TABLE 9
Samples Generated and Analyzed

| | | | XRPD |
|---|---|---|---|
| Method | Solvent^{a} | Conditions^{b} | Pattern^{c} |
| cooling | acetone | 60° C. → RT | A |
| | acetonitrile | 80° C. → RT | A |
| | DCM | 40° C. → 0° C. | A |
| | 1,4-dioxane | 100° C. → 0° C. | A |
| | DMF | 155° C. → 0° C. | A |
| | ethanol (absolute) | 80° C. → 0° C. | A |
| | ethyl acetate | 80° C. → 0° C. | A |
| | diethyl ether | 35° C. → 0° C. | A |
| | MeOH | 65° C. → RT | A |
| | MEK | 80° C. → 0° C. | A |
| | 2-MeTHF | 80° C. → RT | A + B |
| | 2-propanol | 80° C. → RT | A + B |
| | tetrahydrofuran | 65° C. → RT | A |
| | toluene | 110° C. → RT | A |
| evaporation | DMF | open vial, RT | A + B |
| | 1,4-dioxane | open vial, RT | A |
| | EtOH | open vial, RT | A + B |
| | MeOH | open vial, RT | A + B |
| | 2-MeTHF | open vial, RT | A + B |
| | 2-PrOH | open vial, RT | A + B |
| | THF | open vial, RT | A |
| | acetone/water (95/5) | open vial, RT | A + B |
| | ACN/water (95/5) | open vial, RT | A + B |
| | EtOH/water (95/5) | open vial, RT | A + B |
| | MeOH/water (95/5) | open vial, RT | A + B |
| | THF/water (95/5) | open vial, RT | A |

^{a}ACN = acetonitrile; DCM = dichloromethane; DMF = N,N-dimethylformamide; EtOH = absolute ethanol; MeOH = methanol; MEK = methyl ethyl ketone; 2-PrOH = 2-propanol; THF = tetrahydrofuran
^{b}AS = anti-solvent; NC = no crystallization; RT = room temperature
^{c}NC = non-crystalline

TABLE 10
Samples Generated and Analyzed

| | | | XRPD |
|---|---|---|---|
| Method | Solvent^{a} | Conditions^{b} | Pattern^{c} |
| milling | acetone | grind, 20 mins | A |
| | acetonitrile | grind, 20 mins | A |
| | DCM | grind, 20 mins | A |
| | 1,4-dioxane | grind, 20 mins | A |
| | DMF | grind, 20 mins | A |
| | ethanol (absolute) | grind, 20 mins | A |
| | ethyl acetate | grind, 20 mins | A |
| | diethyl ether | grind, 20 mins | A |
| | MeOH | grind, 20 mins | A |
| | MEK | grind, 20 mins | A |
| | 2-MeTHF | grind, 20 mins | A |
| | 2-propanol | grind, 20 mins | A |
| | tetrahydrofuran | grind, 20 mins | A |
| | toluene | grind, 20 mins | A |
| | water | grind, 20 mins | A |
| | none | grind, 20 mins | A |
| slurry | acetone | RT, 7 days | A |
| | acetonitrile | RT, 7 days | A |
| | DCM | RT, 7 days | A |
| | 1,4-dioxane | RT, 7 days | A |
| | DMF | RT, 7 days | LC |
| | ethanol (absolute) | RT, 7 days | A |
| | ethyl acetate | RT, 7 days | A + B |
| | diethyl ether | RT, 7 days | A |
| | MeOH | RT, 7 days | A |
| | MEK | RT, 7 days | A + B |
| | 2-MeTHF | RT, 7 days | A |
| | 2-propanol | RT, 7 days | A |
| | tetrahydrofuran | RT, 7 days | A |
| | toluene | RT, 7 days | A |
| | water | RT, 7 days | A + B |
| | | RT, 2 days, wet | A |
| | acetone/water (95/5) | RT, 7 days | A + B |
| | ACN/water (95/5) | RT, 7 days | A |
| | EtOH/water (95/5) | RT, 7 days | A |
| | MeOH/water (95/5) | RT, 7 days | A |
| | 2-PrOH/water (95/5) | RT, 7 days | A + B |
| | THF/water (95/5) | RT, 7 days | A |

^{a}ACN = acetonitrile; DCM = dichloromethane; DMF = N,N-dimethylformamide; EtOH = absolute ethanol; MeOH = methanol; MEK = methyl ethyl ketone; 2-PrOH = 2-propanol; THF = tetrahydrofuran
^{b}AS = anti-solvent; NC = no crystallization; RT = room temperature
^{c}LC = low crystallinity, NC = non-crystalline

TABLE 11
Samples Generated and Analyzed

| | | | XRPD |
|---|---|---|---|
| Method | Solvent^{a} | Conditions^{b} | Pattern^{c} |
| precipitation | DMF | −15° C., acetone AS | A + B |
| | | −15° C., DCM AS | A |
| | | −15° C., EtOAc AS | A + B |
| | | −15° C., Et₂O AS | A + B |
| | | −15° C., toluene AS | A + B |
| | | 5° C., water AS | A |
| | MeOH | −15° C., acetone AS | A + B(tr) |
| | | −15° C., DCM AS | A + B |
| | | −15° C., Et₂O AS | A |
| | | −15° C., toluene AS | C |
| | | | C |
| | | 5° C., water AS | A + B |
| | THF | −15° C., acetone AS | A |
| | | −15° C., DCM AS | A + B(tr) |
| | | −15° C., hex AS | A |
| | | −15° C., Et₂O AS | A + B |
| | | 5° C., water AS | B + A (tr) |
| | | | B + A (tr) |
| vapor | MeOH | RT, acetone AS | A |
| diffusion | | RT, DCM AS; evap | A (LC) |
| | | RT, Et₂O AS | A |
| | | RT, MEK AS | A |
| | THF | RT, acetone AS | A |
| | | RT, DCM AS | A |
| | | RT, EtOAc AS | A |
| | | RT, Et₂O AS | A + B(tr) |
| | | RT, hex AS | A + B(tr) |
| | acetone/water (95/5) | RT, DCM AS; evap | A + NC |
| | | RT, EtOAc AS | B + A(tr) |
| | | RT, Et₂O AS; evap | A + B |
| | | RT, hex AS; evap | A |
| heat/humidity | water vapor | RT, 59% RH | A |
| | | RT, 75% RH | A |
| | | RT, 97% RH | A |
| | | 40° C., 75% RH | A |
| | none | RT, 0% RH | A |

^{a}ACN = acetonitrile; DCM = dichloromethane; DMF = N,N-dimethylformamide; EtOH = absolute ethanol; MeOH = methanol; MEK = methyl ethyl ketone; 2-PrOH = 2-propanol; THF = tetrahydrofuran
^{b}AS = anti-solvent; NC = no crystallization; RT = room temperature
^{c}LC = low crystallinity, tr = trace

As disclosed herein, three polymorphs have been identified, designated as forms A, B, and C. An overlay plot is shown in FIG. 6. Each form was analyzed by thermal analysis (Table 12) and NMR (Table 13).

TABLE 12
Thermal Analysis of Polymorphs

| Form | Results |
|---|---|
| B | endo 117.79° C. |
| | 4.30% start to 125° C. (1.1 moles water) |
| C | endo 169.52° C. |
| | 0.12% start to 150° C. |

TABLE 13
NMR Analysis of Polymorphs

| Form | Results |
|---|---|
| A + B | consistent with a 1:1 |
| | (API:acid) salt |
| B | consistent with a 1:1 |
| | (API:acid) salt |
| C | consistent with a 1:1 |
| | (API:acid) salt |
| | residual methanol and toluene |

Competitive slurry experiments were performed to determine the most stable form at various conditions (Table 14). Forms A, B and C were slurried in ethyl acetate, MEK, and (95:5) acetone/water at various temperatures.

TABLE 14
Competitive Slurry Experiments

| Starting | | | XRPD |
|---|---|---|---|
| Material | Solvent | Conditions | Pattern |
| A and B | EtOAc | 5° C., 5 days | A |
| | | 40° C., 2 days | A |
| | MEK | 5° C., 5 days | A |
| | | 40° C., 2 days | A |
| | (95:5) | 5° C., 5 days | A + B |
| | acetone/water | 40° C., 2 days | A |
| A, B, C | EtOAc | 5° C., 6 days | |
| | | RT, 6 days | |
| | | 40° C., 2 days | A |
| | MEK | 5° C., 6 days | |
| | | RT, 6 days | |
| | | 40° C., 2 days | A |
| | (95:5) | 5° C., 6 days | |
| | acetone/water | RT, 6 days | |
| | | 40° C., 2 days | A + B (tr) |

### Single Crystal Structure of Esreboxetine Succinate

Following the polymorph screens for esreboxetine fumarate, the crystal structure of esreboxetine succinate was solved and confirmed to be the (S,S)-stereoisomer.

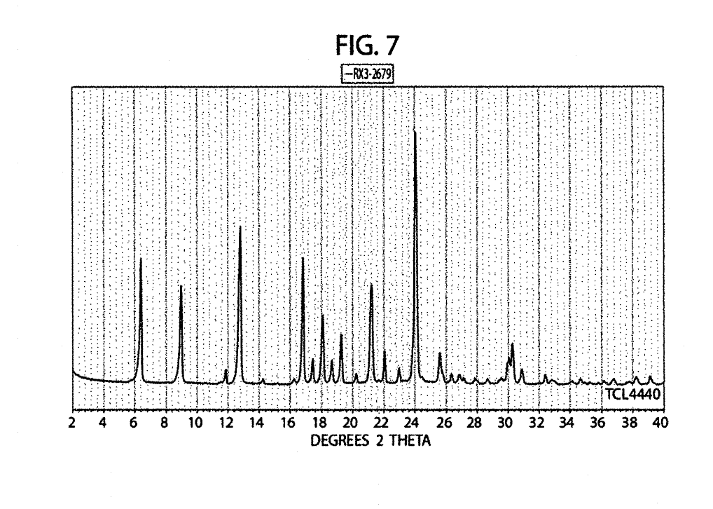

A sample of esreboxetine succinate was analyzed by x-ray powder diffraction (XRPD), which showed it to be crystalline (FIG. 7).

Attempts were made to grow single crystals of esreboxetine succinate of sufficient quality and size for x-ray structure determination. The crystallization experiment that was carried out is listed in Table 11. The crystals produced were examined by optical microscopy and appeared to be of sufficient size and quality. The sample was submitted to the crystallographer at Purdue University, who mounted one crystal, collected diffraction data, and solved the structure.

TABLE 15
Single Crystal Growth Experiments

| Method | Solvent | Conditions | Observations |
|---|---|---|---|
| cooling | ethanol | reflux → ambient | singles |

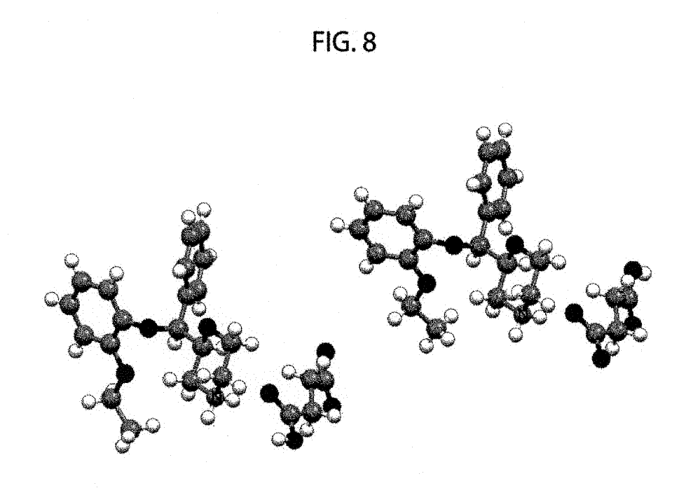

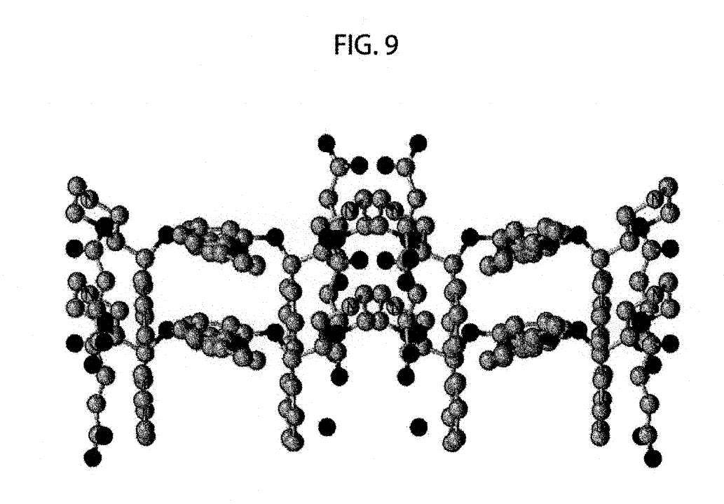

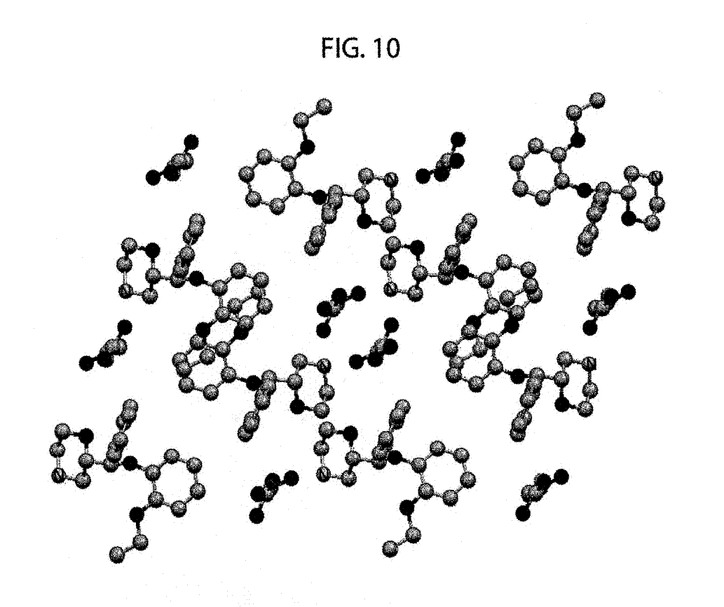

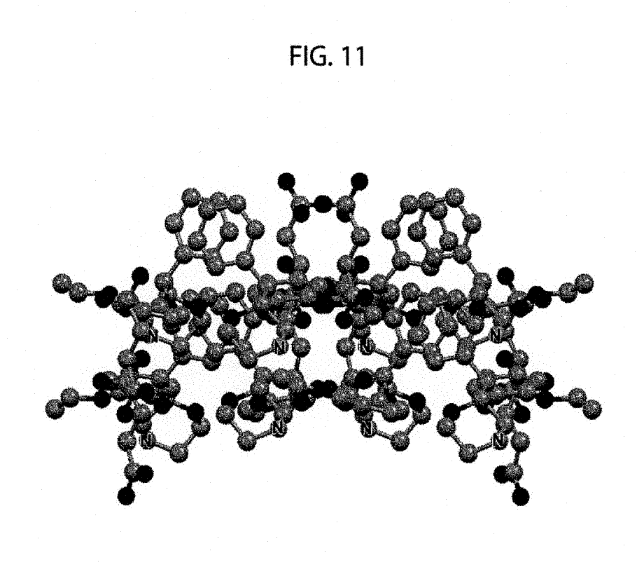

The asymmetric unit and packing diagrams obtained from the structural data are shown in FIG. 8 through FIG. 11.

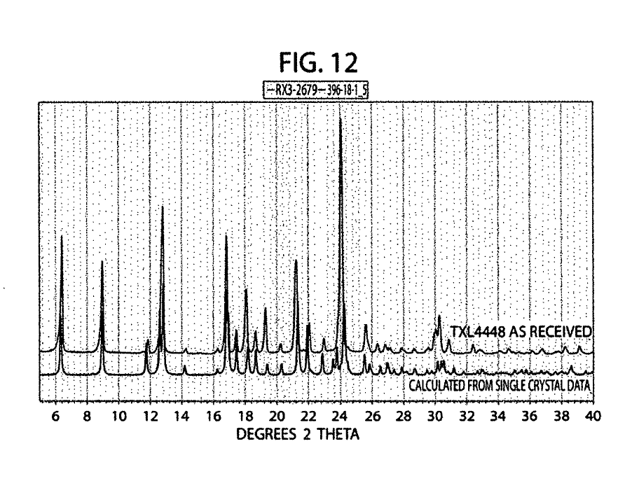

An overlay plot of the measured XRPD pattern of the starting material and the pattern calculated from the single-crystal data is shown in FIG. 12.

### Experimental Methods Employed in Salt and Polymorph Screening

### X-Ray Powder Diffraction (XRPD):

The Rigaku Smart-Lab X-ray diffraction system was configured for reflection Bragg-Brentano geometry using a line source X-ray beam. The x-ray source is a Cu Long Fine Focus tube that was operated at 40 kV and 44 ma. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40° 2θ using a continuous scan of 6°2θ per minute with an effective step size of 0.02° 2θ.

### Differential Scanning Calorimetry (DSC):

DSC analyses were carried out using a TA Instruments Q2000 instrument. The instrument temperature calibration was performed using indium. The DSC cell was kept under a nitrogen purge of ˜50 mL per minute during each analysis. The sample was placed in a standard, crimped, aluminum pan and was heated from 25° C. to 350° C. at a rate of 10° C. per minute.

### Thermogravimetric (TG) Analysis:

The TG analysis was carried out using a TA Instruments Q50 instrument. The instrument balance was calibrated using class M weights and the temperature calibration was performed using alumel. The nitrogen purge was ˜40 mL per minute at the balance and ˜60 mL per minute at the furnace. Each sample was placed into a pre-tared platinum pan and heated from 20° C. to 350° C. at a rate of 10° C. per minute.

### Dynamic Vapor Sorption (DVS) Analysis:

DVS analyses were carried out TA Instruments Q5000 Dynamic Vapor Sorption analyzer. The instrument was calibrated with standard weights and a sodium bromide standard for humidity. Samples were analyzed at 25° C. with a maximum equilibration time of 60 minutes in 10% relative humidity (RH) steps from 5 to 95% RH (adsorption cycle) and from 95 to 5% RH (desorption cycle).

### Nuclear Magnetic Resonance (NMR) Spectroscopy:

The ¹H NMR spectra were acquired on a Bruker DRX-500 spectrometer located at the Chemistry Department of Purdue University. Samples were prepared by dissolving material in DMSO-d₆. The solutions were filtered and placed into individual 5-mm NMR tubes for subsequent spectral acquisition. The temperature controlled (298K)¹H NMR spectra acquired on the DRX-500 utilized a 5-mm cryoprobe operating at an observing frequency of 499.89 MHz.

### Preparation of Esreboxetine Fumarate:

A solution of 150.0 mg of esreboxetine in 2 mL of acetone was combined with a solution of 55.7 mg of fumaric acid in 7 mL of acetone. The resulting solution was transferred to the freezer at −15° C. temperature. Precipitation occurred after three days. Centrifugation and air-drying of the remaining solid afforded 177.8 mg (86% yield). The solid was analyzed by XRPD.

### Typical Polymorph Screen Evaporation Experiment

A solution of 16.4 mg of salt in ½ mL of acetone was placed in an open vial in a fume hood for one day. The acetone evaporated, leaving solid that was analyzed by XRPD.

### Typical Polymorph Screen Cooling Experiment:

A vial was charged with 18.4 mg of salt. The vial was placed on a hot plate set at reflux. Absolute ethanol was added until the solid dissolved; about 5 mL. The resulting solution was allowed to cool to room temperature, during which time crystallization did not occur. The vial was placed in a refrigerator (about 5° C.) overnight, during which time crystallization did not occur. The vial was placed in a freezer (about −15° C.) for twelve days, during which time crystallization occurred. The solvent was decanted and the solid dried in the air and analyzed by XRPD.

### Typical Polymorph Screen Slurry Experiment:

A slurry of 19.7 mg of salt in 500 μL of 2-propanol was stirred at ambient temperature for seven days. The solvent was decanted and the solid dried in the air and analyzed by XRPD.

### Typical Polymorph Screen Precipitation Experiment:

A solution of 18.8 mg of salt in 800 μL of acetone was treated with 4 mL of cold ethyl ether. Crystallization did not occur immediately so the solution was placed in a freezer (about −15° C.) overnight, during which time crystallization occurred. The solvent was decanted and the solid dried in the air and analyzed by XRPD.

### Typical Polymorph Screen Stress Experiment:

About 15.8 mg of salt was placed in an open vial and the vial was placed in a saturated salt chamber at 97% relative humidity. After about seven days the sample was analyzed by XRPD.

### Typical Polymorph Screen Grinding Experiment:

A mixture of 16.8 mg of salt was 10 μL of 2-propanol was placed in a PEEK grinding cup with a steel ball. The cup was placed in a Retsch mill and agitated at 100% power for about twenty minutes. The resulting solid was analyzed by XRPD.

### Indications for Use

Esreboxetine fumarate and other salt iterations of esreboxetine and their respective polymorphs will be reviewed and developed for the indication of the treatment of fibromyalgia, pending FDA approval. Two separate trials have shown esreboxetine to be an efficacious SNRI in the treatment of fibromyalgia. The various salts of esreboxetine proposed here, in particular esreboxetine fumarate, are expected to act in a similar manner and to have improved formulation properties.

### REFERENCES

1. Anderberg U M, Marteinsdottir I, von Knorring L. Citalopram in Patients with Fibromyalgia—A Randomized, Double-Blind, Placebo-Controlled Study. Eur J Pain. 2000; 4: 27-35.
2. Arnold L M, Keck P E, Welge J A. Antidepressant Treatment of Fibromyalgia: A Meta-Analysis and Review. Psychosomatics. 2000; 41: 104-113.
3. Arnold L M, Hess E V, Hudson J I, et al. A Randomized, Placebo-Controlled, Double-Blind, Flexible-Dose Study of Fluoxetine in the Treatment of Women with Fibromyalgia. Am J Med. 2002; 112: 191-197.
4. Arnold L M, Lu Y, Crofford L J, et al. A Double-Blind, Multicenter Trial Comparing Duloxetine with Placebo in the Treatment of Fibromyalgia Patients with or without Major Depressive Disorder. Arthritis Rheum. 2004; 50: 2974-2984.
5. Arnold L M, Rosen A, Pritchett Y L, et al. A Randomized, Double-Blind, Placebo-Controlled Trial of Duloxetine in the Treatment of Women with Fibromyalgia with or without Major Depressive Disorder. Pain. 2005; 119: 5-15.
6. Arnold L M, Chatamra K, Hirsch I, Stoker M. Safety and Efficacy of Esreboxetine in Patients with Fibromyalgia: An 8-Week, Multicenter, Randomized, Double-Blind, Placebo-Controlled Study. Clinical Therapeutics. 2010; 32(9):1618-1632.
7. Arnold L M, Hirsch I, Sanders P, Ellis A, Hughes B. Safety and Efficacy of Esreboxetine in Patients With Fibromyalgia: A Fourteen-Week, Randomized, Double-Blind, Placebo-Controlled, Multicenter Trial. Arthritis and Rheumatism. 2012; 64(7): 2387-2397.
8. Baldwin D S, Buis C, Carabal E (2000) Rev Contemp Pharmacother 11:321
9. Hajos M, Fleishaker J C, Filipiak-Reisner J K, Brown M T, Wng EHF (2004) CNS Drug Rev 10:23
10. Berger A, Dukes E, Martin S, Edelsberg J, Oster G. Characteristics and Healthcare Costs of Patients with Fibromyalgia Syndrome. International Journal of Clinical Practice. 2007; 61(9): 1498-1508.
11. Blumenthal D E, Malemud C J. Recent Strategies for Drug Development in Fibromyalgia Syndrome. Expert Review of Neurotherapeutics. 2016.
12. Bohn L M, Gainetdinov R R, Caron M G. Potentiated Opioid Analgesia in Norepinephrine Transporter Knock-Out Mice. J Neurosci. 2000; 20: 9040-9045
13. Fishbain D A, Cutler R, Rosomoff H L, Rosomoff R S. Evidence-Based Data from Animal and Human Experimentation Studies on Pain Relief with Antidepressants: A Structured Review. Pain Med. 2000; 1: 310-316.
14. Gendreau R M, Thorn M D, Gendreau J F, et al. Efficacy of Milnacipran in Patients with Fibromyalgia. J Rheumatol. 2005; 32: 1975-1985.
15. Goldenberg D, Mayskiy M, Mossey C, et al. A Randomized, Double-Blind, Crossover Trial of Fluoxetine and Amitriptyline in the Treatment of Fibromyalgia. Arthritis Rheum. 1996; 39: 1852-1859.
16. Millan M J. Descending Control of Pain. Prog Neurobiol. 2002; 66: 355-474.
17. Walitt B, Nahin R L, Katz R S, Bergman M J, Wolfe F. The Prevalence and Characteristics of Fibromyalgia in the 2012 National Health Interview Survey. PLoS ONE. 2015; 10(9): 1-16.
18. Wolfe F, Ross K, Anderson J, Russel L I, Hebert L. The Prevalence and Characteristics of Fibromyalgia in the General Population. Arthritis and Rheumatism. 1995; 38(1): 19-28.
19. Vincent A, Lahr B D, Wolfe F, et al. Prevalence of Fibromyalgia: A Population-Based Study in Olmsted County Minnesota, Utilizing the Rochester Epidemiology Project. Arthritis Care Res. 2013; 65(5): 786-792.

The preceding is a detailed description of particular embodiments/aspects of the disclosure. It will be appreciated that, although specific embodiments/aspects of the disclosure have been described herein for purposes of illustration, various modifications may be made without departing from the spirit and scope of the present disclosure. Accordingly, the disclosure herein is not limited except as by the appended claims.

What is claimed is:

1. A salt of (2S)-2-[(S)-(ethoxyphenoxy)phenylmethyl]morpholine (esreboxetine) as shown in Formula I: 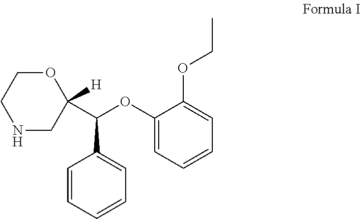 [SMILES: CCOc1ccccc1O[C@@H](c1ccccc1)[C@@H]1CNCCO1]
wherein the salt is esreboxetine fumarate.

2. The salt according to claim 1, wherein the esreboxetine fumarate is crystalline.

3. The salt of claim 2, wherein the salt is anhydrous crystalline esreboxetine fumarate Form A, B, C and/or a combination thereof.

4. The salt of claim 2, wherein the salt is hydrated crystalline esreboxetine fumarate Form A, B, C and/or a combination thereof.

5. A pharmaceutical composition comprising the salt esreboxetine fumarate with a pharmaceutically acceptable carrier, diluent or excipient.

6. The pharmaceutical composition of claim 5, wherein the salt is anhydrous crystalline esreboxetine fumarate Form A, B, C and/or a combination thereof.

7. The pharmaceutical composition of claim 5, wherein the salt is hydrated crystalline esreboxetine fumarate Form A, B, C and/or a combination thereof.

8. The anhydrous esreboxetine fumarate crystalline Form A of claim 3, characterized in that the crystalline form has an X-ray diffraction pattern (XRPD) comprising at least one peak at about 7.0 degrees 2θ.

9. The anhydrous esreboxetine fumarate crystalline Form A of claim 8, wherein said crystalline form exhibits an XRPD pattern further comprising at least one peak selected from the group consisting of about 6.5 and 8.9 degrees 2θ.

10. The compound of claim 9, wherein said crystalline Form A exhibits an XRPD pattern further comprising at least one peak selected from the group consisting of about 12.5, 16.5, 17.9, 18.2, 21.0, and 24.0 degrees 2θ.

11. The anhydrous esreboxetine fumarate crystalline Form B of claim 3, characterized in that the crystalline form has an X-ray diffraction pattern (XRPD) comprising at least one peak at about 5.9 degrees 2θ.

12. The anhydrous esreboxetine fumarate crystalline Form B of claim 11, wherein said crystalline form exhibits an XRPD pattern further comprising at least one peak selected from the group consisting of about 11.5 and 17.2 degrees 2θ.

13. The compound of claim 12, wherein said crystalline Form B exhibits an XRPD pattern further comprising at least one peak selected from the group consisting of about 17.9, 20.0, and 23.2 degrees 2θ.

14. The anhydrous esreboxetine fumarate crystalline Form C of claim 3, characterized in that the crystalline form has an X-ray diffraction pattern (XRPD) comprising at least one peak at about 6.5 degrees 2θ.

15. The anhydrous esreboxetine fumarate crystalline Form C of claim 14, wherein said crystalline form exhibits an XRPD pattern further comprising at least one peak selected from the group consisting of about 13.0 and 13.4 degrees 2θ.

16. The compound of claim 15, wherein said crystalline Form C exhibits an XRPD pattern further comprising at least one peak selected from the group consisting of about 14.8, 15.2, 18, 18.5, 19.2, 20.0, 21.0, 22.4, and 23.5 degrees 2θ.

17. The compound of claim 3, wherein said crystalline Form A exhibits an XRPD pattern substantially the same as pattern A of FIG. 6.

18. The compound of claim 3, wherein said crystalline Form B exhibits an XRPD pattern substantially the same as pattern B of FIG. 6.

19. The compound of claim 3, wherein said crystalline Form C exhibits an XRPD pattern substantially the same as pattern C of FIG. 6.

20. The anhydrous crystalline esreboxetine fumarate Form A of claim 3, characterized by at least one of:
(a) an XPRD pattern exhibiting at least four of the peaks shown in pattern A of FIG. 6; and
(b) an NMR spectrum substantially the same as FIGS. 13 and 14.

21. The anhydrous crystalline esreboxetine fumarate Form B of claim 3, characterized by at least one of:
(a) an XPRD pattern exhibiting at least four of the peaks shown in pattern B of FIG. 6;
(b) an NMR spectrum substantially the same as FIGS. 17 and 18.

22. The anhydrous crystalline esreboxetine fumarate Form C of claim 3, characterized by at least one of:
(a) an XPRD pattern exhibiting at least four of the peaks shown in pattern C of FIG. 6; and
(b) an NMR spectrum substantially the same as FIGS. 19 and 20.

23. The anhydrous crystalline esreboxetine fumarate Form A+B of claim 3, characterized by an NMR spectrum substantially the same as FIGS. 15 and 16.
